(19) United States
(12) Patent Application Publication
(10) Pub. No.: US 2022/0335112 A1
(43) Pub. Date: Oct. 20, 2022

(54) BIOMETRIC HEALTH CREDENTIALS

(71) Applicant: Adrian Gropper, Watertown, MA (US)

(72) Inventor: Adrian Gropper, Watertown, MA (US)

(21) Appl. No.: 17/723,272

(22) Filed: Apr. 18, 2022

Related U.S. Application Data
(60) Provisional application No. 63/176,130, filed on Apr. 16, 2021.

(51) Int. Cl. G06F 21/32; G16H 10/60

(52) U.S. Cl. CPC G06F 21/32; G16H 10/60

(57) ABSTRACT

Disclosed, in one general aspect, is a credential for conveying verified information about an individual. The credential includes a biometric for the individual that is both sufficiently human-recognizable as to enable a human to match the individual with the biometric, and reliably machine-readable to enable the biometric to be reliably acquired by a computer sensor. The credential also includes a machine-readable verification code to verify against the biometric. Methods and systems for creating, validating, and verifying credentials are also presented.

### CROSS-REFERENCE TO RELATED APPLICATION

This application claims the benefit of U.S. Provisional Application No. 63/176,130, filed Apr. 16, 2021, which is herein incorporated by reference.

### FIELD OF THE INVENTION

This invention relates to methods and apparatus for conveying verified information about an individual.

### BACKGROUND OF THE INVENTION

Converting the COVID CDC Vaccination Card into a standardized digital credential is turning out to be harder than expected. The conversation has become prominent in the news and risks being politicized to the detriment of public health efforts around the world.

The core problem is a combination of privacy and equity. Paper vaccination credentials tend to be only loosely linked to a verified identity like a driver's license. Many vaccination or test sites do not ask for proof of identity and proponents of digital credentials apparently like to talk about how easy it is to buy fake cards.

The privacy and equity problem is aggravated by the range of contexts where the credentials are to be verified. These range from totally voluntary to essential: from restaurant, to travel, to school, to employment.

Behind the scenes, there are a handful of groups with different agendas promoting their digital solutions to the privacy and equity problems. Their common denominator is a QR code that can be voluntarily presented on paper, like the current CDC card, or on a smartphone. It's assumed that the presentation will have name and DOB and some have a photo as well.

Introducing a biometric into the digital credential to prevent fraud is potentially a privacy issue if the biometric is centralized, as in a drivers license authority or Aadhaar in India.

### SUMMARY OF THE INVENTION

In one general aspect, the invention features a credential for conveying verified information about an individual. The credential includes a biometric for the individual that is both sufficiently human-recognizable as to enable a human to match the individual with the biometric, and reliably machine-readable to enable the biometric to be reliably acquired by a computer sensor. The credential also includes a machine-readable verification code to verify against the biometric.

In preferred embodiments, the credential can be a paper credential that includes an image of the individual and a signed hash value derived from the image. The credential can include an image of the individual and a signed hash value derived from the image. The can be quantized using a quantization function. The credential can further include a certificate indicating that the individual has been subject to a medical procedure. The credential can further include a certificate indicating that the individual has been vaccinated against a particular pathogen. The credential can further include a certificate indicating that the individual has been tested for a particular medical condition.

In another general aspect, the invention features a credential verification method for verifying information about an individual that includes receiving visual access to a biometric for the individual associated with a credential to enable a human to match the individual with the biometric, acquiring the biometric associated with the credential, acquiring a verification code associated with the credential, and verifying that the acquired biometric matches the acquired verification code.

In preferred embodiments, the method can further include verifying signed information about the individual associated with the credential. The method can further include verifying signed information indicating that the individual associated with the credential has been subject to a medical procedure. The step of verifying can be performed anonymously. The verifying can be performed independent of any matching of information about the individual with information stored outside of the credential.

In a further general aspect, the invention features a credential verification method for verifying information about an individual, including incorporating into the credential a biometric for the individual that is both sufficiently human-recognizable as to enable a human to match the individual with the biometric, and reliably machine-readable to enable the biometric to be reliably acquired by a computer sensor. The method also includes incorporating into the credential a machine-readable verification code to verify against the biometric.

In preferred embodiments, the method can further include quantizing an image of the individual to produce the biometric. The method can further include receiving the quantized image of the individual and the verification code from the individual. The method can further include incorporating further information about the individual into the credential. The method can further include revoking the credential. The credential can be devoid of information that identifies who the individual is. The method can further include receiving visual access to the biometric for the individual associated with the credential to enable a human to match the individual with the biometric, acquiring the biometric associated with the credential, acquiring the verification code associated with the credential, and verifying that the acquired biometric matches the acquired verification code. The incorporating of the verification code and the verifying that the acquired biometric matches the acquired verification code can be performed according to an open-source procedure.

In another general aspect, the invention features a system for creating a credential for conveying verified information about an individual that includes a biometric interface for obtaining a biometric for the individual that is both sufficiently human-recognizable as to enable a human to match the individual with the biometric, and reliably machine-readable to enable the biometric to be reliably acquired by a computer sensor. The system also includes an app responsive to the biometric interface that is operative to derive a machine-readable verification code to verify against the biometric. In preferred embodiments the system can be implemented with a smartphone with the biometric interface including a digital camera.

In a further general aspect, the invention features a system for verifying a credential for conveying verified information about an individual that includes at least one acquisition interface operative to acquire a biometric associated with the credential, and a verification code associated with the credential. The system also includes an app responsive the interface and operative to verify that the acquired biometric matches the acquired verification code. In preferred embodiments the system can be implemented with a smartphone with the acquisition interface including a digital camera.

In one embodiment a quantized face that is still human-verifiable is added to a digital credential presentation. A hash of the quantized face, but not the face itself, would be signed and verifiable as part of the digital credential represented by the QR code.

Systems according to the invention can increase the security of digital credentials, such as vaccination credentials, without compromising the privacy of the human subject of the credential. In one general aspect, a method of enhancing a signed digital document is described by the addition of a human recognizable face image that is also reproducibly converted to a digital code, with this code being part of the signed digital document. Presentation of the enhanced document to a human inspector allows them to visually verify that the image matches the presenter and, using common digital means such as a smartphone, to verify that the signed digital document refers to the same image. The method to re-convert a human-recognizable likeness to an exact digital code in a readily reproducible manner can result in a signed digital document that is much smaller than if the digitized image itself had to be included in the document.

### BRIEF DESCRIPTION OF THE DRAWING

FIG. 1 is a monochrome rendering of a color digital document with a human-recognizable component according to the invention;

FIG. 2 is an enlarged monochromatic rendering of the human-recognizable component of FIG. 1;

FIG. 3 is a partial list of the digital values of tiles in the human-recognizable component of FIG. 2;

FIG. 4 is a flow diagram showing the use of digital credential standards;

FIG. 5 is a block diagram of an embodiment of a credential validation system according to the invention; and

FIG. 6 is a block diagram of an embodiment of a credential verification system according to the invention.

### DETAILED DESCRIPTION OF AN ILLUSTRATIVE EMBODIMENT

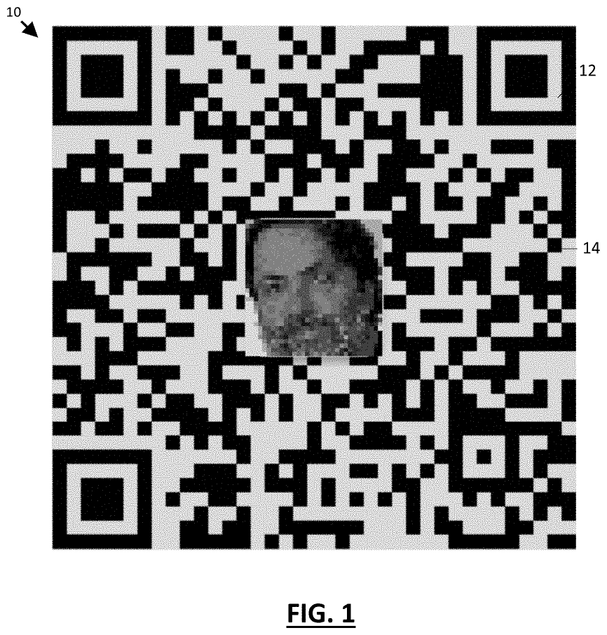

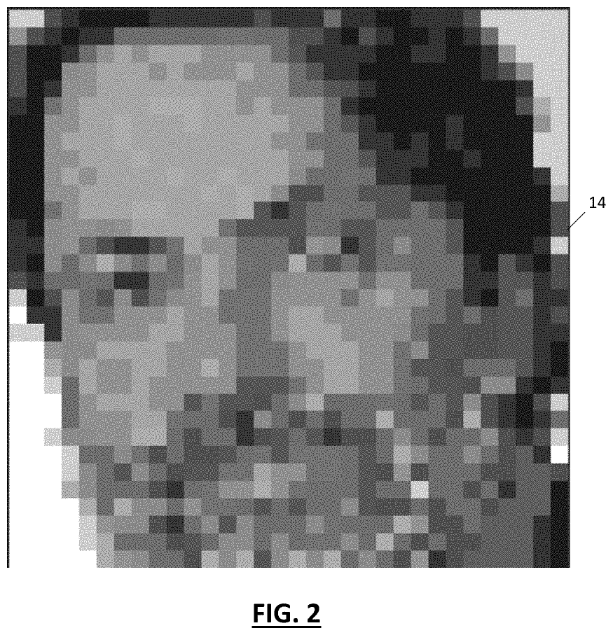

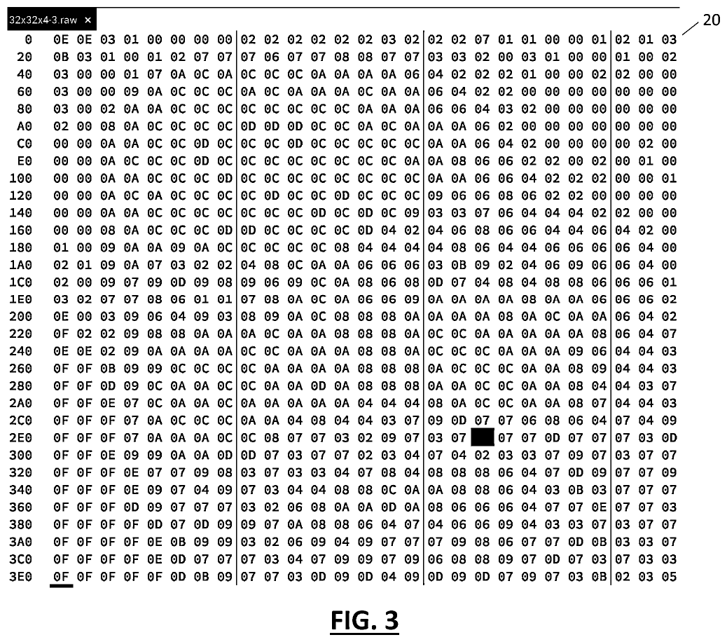

Referring to FIGS. 1-3, an illustrative embodiment of a digital document 10 according to the invention includes a human-recognizable component 14, such as a digital image of a patient's face, and a verification code, such as a QR code. The document can be presented in different ways, such as on paper or on a device screen, such as a smartphone screen.

In this embodiment, GIMP was used to reduce the resolution of the image to 32×32 pixels without interpolation and then indexed to 16 colors. The idea is that any camera that can read a QR code can also reliably recover the 16 colors with zero errors so the hash can be matched with the hash in the QR code credential. The choice of a 512 Byte template is just an example.

The verifier uses a mobile app that combines four functions:
A typical QR code API returns the digital credential (directly or by reference).
A typical camera API returns the quantized photo.
A clustering algorithm, like a trivial JPEG compression algorithm, recovers the 16 indexed colors in the original and calculates the hash of the quantized photo.
A typical signature verification algorithm compares the locally calculated hash with the one in the signed digital credential and reports Success or Fail.

A smartphone app is used at the issuer to produce the QR code. That app also needs a face photo, as above, to quantize and hash and add to the credential before signing.

It's important to recognize that, once the digital credential is handed or sent to the patient, the issuer does not need to store either the original or the quantized image.

Privacy sensitive patients that don't trust the issuer with any biometric have the option of taking their own selfie and using a quantizer app so they can deliver the hash back to the issuer to sign and then verify. A failed verification results in immediate revocation of the credential. Variations on this semi-self-issue option are possible if revocation is not available.

According to another important privacy feature, other credentials issued to the same patient could use a new face photo each time with different lighting and perspective so the hash of the quantized face would be different (<512 Bytes of entropy in the example, but still significant). This avoids issuers or verifiers colluding to correlate across credentials on the basis of the hash.

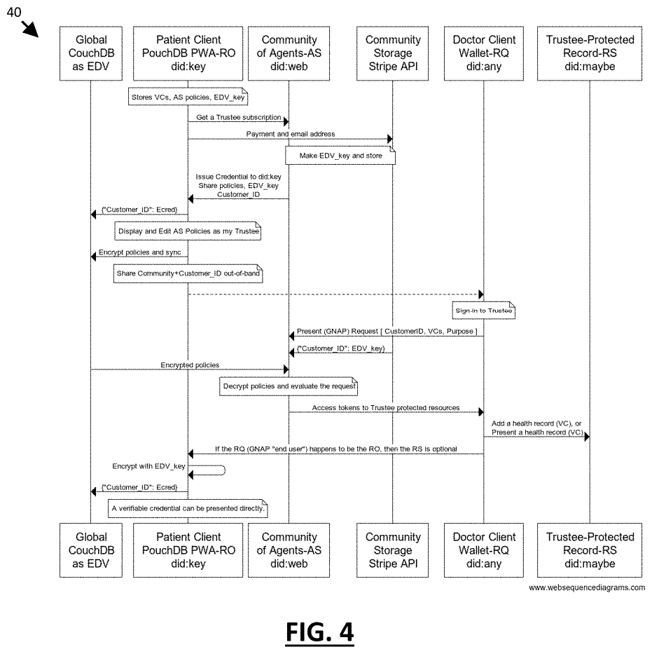

Referring to FIG. 4, for example, the health record credential could be leveraged by the well-known digital identifier (W3C DID), verifiable credentials (W3C VC) and delegated authorization (IETF GNAP) standards, which are herein incorporated by reference.

Table 1. presents a summary of concerns with digital credentials.

TABLE 1

| | Challenges for VaxCreds | Solutions |
|---|---|---|
| Standards | Wait for ‘common standard’ | No standards required for QR code in short |
| | to emerge for issuance so | term. Transfer complexity to free universal |
| | that verification can become | scanning apps that can read any QR vaxcred |
| | simple | |
| Ecosystems | Integration complexity with | Treat vaxcreds as one-way offline output |
| | external ecosystem of | with no linkage back to EHR/Blockchain |
| | Blockchain, EHR, | for verification. With no reverse linkage, no |
| | Identity | PII audit trail possible. |
| | | No additional user data collection required. |
| Biz Model | Companies pushing models | Use offline-offline solution, nearly free to |
| | with cloud services, pay-per- | everyone. States can pay for app. |
| | verification, exploit | |
| | streaming data | |
| Equity | Govt insist equity, no | Paper-first approach, selective disclosure |
| | smartphones, no PII for | with initials/year (not full PII), |
| | vulnerable population | |

### Example: Inspection Processing Sequence

On a normal (e.g., $300+) smartphone
Install an app (a PWA might be even better)
That uses the available QR code API to return a link to a custodial credential
That also uses the camera API to capture an image
   Interactively positioned the way a check deposit image is done
Digital image is converted to a regular 32×32 grid
   Represented as 1,024 values in a list
The list is subject to a clustering algorithm looking for 16 channels
Replace each value in the list with the most likely 4-bit channel identifier
Calculate a hash of the 512 Byte list
Compare the hash with one stored in the digital credential retrieved via QR
Display a Green Check or Red X.

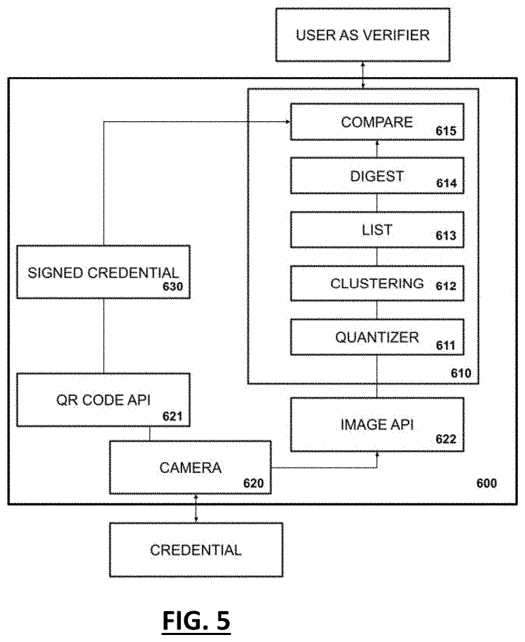

Referring to FIG. 5, an illustrative embodiment of a credential validation system according to the invention can be implemented with a digital processor 600 that includes a camera 620 with an interface that interprets a bar code 621 as a link to a signed credential 630 as well as an interface to the image itself 622. Validation app 610 decodes the facial image part of 622 into the digest 615 which must match the corresponding entry in the signed credential 630. Validation app 610 applies a quantizer 611 on the predetermined image grid, a clustering of the quantized values 612, creating a list 613, which is turned into a digest or hash 614 for validation by comparison in 615.

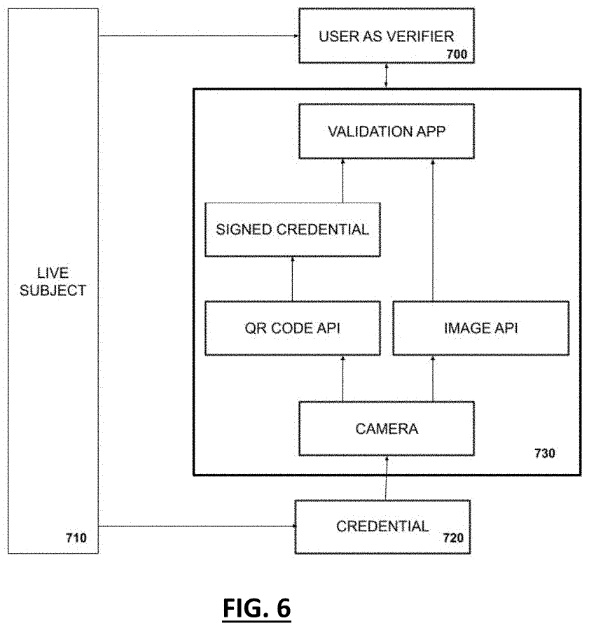

Referring to FIG. 6, an illustrative embodiment of a credential verification system according to the invention can be implemented with a digital processor 730. Verification proceeds when user 700 is presented with a live subject 710 that also provides a credential 720. Verifier 700 uses their digital processor 730 to scan the subject's credential 720. Verifier 700 performs their own validation step by matching the image component of credential 720 with the live subject 710 and, importantly, by ensuring that the validation app 730 is reporting a successful match of the image digest with the corresponding entry in the signed digital credential. This avoids fraud by the subject altering the digital credential or by using a digital credential issued to another person.

A complementary issuance operation is easily derived from the verification method. A modified version of the Digital Processor with Camera 600 can be used to issue a Digital Credential with Quantized Face 720 at the point of vaccination or other credential inception event.

### Health Equity Concerns

With vaccination credentials as an example, it helps to separate the vocabulary credential components from the identity credential components.

The identity aspects relate to equity and involve access to technology such as smartphones, employment discrimination based on the ability to present and inspect credentials at the worksite, and participation by the undocumented who might endanger themselves and the community if they're reluctant to receive vaccines and tests.

There is some relationship between the vocabulary and identity dimensions, if only because the overall size of the credential is limited by printing and technology cost constraints.

One thing that stands out, for example, is the statement that the patient/subject will be identified by Name and DOB. Would we ever identify someone by Name and License Number or would we ever include a code for Level of Assurance of the identity? Do we allow people to self-identify in order to improve access by the undocumented? Are we setting a precedent for documentation of rapid testing, including home self-testing?

Public health is another concern. Every digital credential issued is an opportunity to collect valuable information on prevalence, side-effects, and demographic disparities. Every digital credential could also be associated with voluntary self-reporting. Getting digital privacy right will have more impact on society than anything having to do with the digital vocabulary conversation.

### References: Facial Imaging

This recent paper covers privacy-related modifications to a facial image.
A Study of Face Obfuscation in ImageNet, arXiv:2103.06191 [cs.CV], Cornell University, Mar. 14, 2021. https://arxiv/org/abs/2103.06191

### References: Vaccine Passports

What Are the Roadblocks to a ‘Vaccine Passport’?
https://www.nytimes.com/2021/04/14/travel/covid-vaccine-passport-excelsior-pass.html
Governor Cuomo Announces Launch of Excelsior Pass to Help Fast-Track Reopening of Businesses and Entertainment Venues Statewide
https://www.governor.ny.gov/news/governor-cuomo-announces-launch-excelsior-pass-help-fast-track-reopening-business- and
https://www.technologyreview/com/2021/04/09/1021934/got-your-covid-shots-you-might-need-vaccine-passport/
https://www.consumerreports/org/personal-information/covid-19-vaccine-data-privacy/
https://epic.org/privacy/medical/coalition-letter-DC-pharmacy-data-collection-040221.pdf
https://gcn.com/articles/2021/05/07/la-wallet-digital-vaccination-record.aspx
Zebra Cards: https://connect.zebra.com/vaccinationcerttification

References: Verifiable Credentials
See also: Proposal: Anchored Resources and Hashlinks for VCs https://github.com/w3c/vc⋅data⋅model/issues/831#issuecomment-960249901
SMART Health Cards https://github.com/smart-on-fhir/health-cards and https://github.com/smart-on-fhir/health-cards/discussions/120
Avatar NFTs https://meebits/larvalabs.com/meebits/list
   All of these reference documents are herein incorporated by reference

The present invention has now been described in connection with a number of specific embodiments thereof. However, numerous modifications which are contemplated as falling within the scope of the present invention should now be apparent to those skilled in the art. Therefore, it is intended that the scope of the present invention be limited only by the scope of the claims appended hereto. In addition, the order of presentation of the claims should not be construed to limit the scope of any particular term in the claims.

What is claimed is:

1. A credential for conveying verified information about an individual, wherein the credential includes:
a biometric for the individual that is both:
sufficiently human-recognizable as to enable a human to match the individual with the biometric, and
reliably machine-readable to enable the biometric to be reliably acquired by a computer sensor, and
a machine-readable verification code to verify against the biometric.

2. The credential of claim 1, wherein the credential is a paper credential that includes an image of the individual and a signed hash value derived from the image.

3. The credential of claim 1, wherein the credential is a credential that includes an image of the individual and a signed hash value derived from the image.

4. The credential of claim 2, wherein the image has been quantized using a quantization function.

5. The credential of claim 1, wherein the credential further includes a certificate indicating that the individual has been subject to a medical procedure.

6. The credential of claim 5, wherein the credential further includes a certificate indicating that the individual has been vaccinated against a particular pathogen.

7. The credential of claim 5, wherein the credential further includes a certificate indicating that the individual has been tested for a particular medical condition.

8. A credential verification method for verifying information about an individual, including:
receiving visual access to a biometric for the individual associated with a credential to enable a human to match the individual with the biometric,
acquiring the biometric associated with the credential,
acquiring a verification code associated with the credential, and
verifying that the acquired biometric matches the acquired verification code.

9. The method of claim 8 further including verifying signed information about the individual associated with the credential.

10. The method of claim 8 further including verifying signed information indicating that the individual associated with the credential has been subject to a medical procedure.

11. The method of claim 8 wherein the step of verifying is performed anonymously.

12. The method of claim 8 wherein the verifying is performed independent of any matching of information about the individual with information stored outside of the credential.

13. A credential verification method for verifying information about an individual, including:
incorporating into the credential a biometric for the individual that is both:
sufficiently human-recognizable as to enable a human to match the individual with the biometric, and
reliably machine-readable to enable the biometric to be reliably acquired by a computer sensor, and
incorporating into the credential a machine-readable verification code to verify against the biometric.

14. The method of claim 13 further including quantizing an image of the individual to produce the biometric.

15. The method of claim 14 further including receiving the quantized image of the individual and the verification code from the individual.

16. The method of claim 13 further including incorporating further information about the individual into the credential.

17. The method of claim 13 further including revoking the credential.

18. The method of claim 13 wherein the credential is devoid of information that identifies who the individual is.

19. The method of claim 13 further including:
receiving visual access to the biometric for the individual associated with the credential to enable a human to match the individual with the biometric,
acquiring the biometric associated with the credential,
acquiring the verification code associated with the credential, and
verifying that the acquired biometric matches the acquired verification code.

20. The method of claim 19 wherein the incorporating of the verification code and the verifying that the acquired biometric matches the acquired verification code are performed according to an open-source procedure.

21. A system for creating a credential for conveying verified information about an individual, wherein the system includes:
a biometric interface for obtaining a biometric for the individual that is both:
sufficiently human-recognizable as to enable a human to match the individual with the biometric, and
reliably machine-readable to enable the biometric to be reliably acquired by a computer sensor, and
an app responsive to the biometric interface that is operative to derive a machine-readable verification code to verify against the biometric.

22. The system of claim 21 wherein the system is implemented with a smartphone and the biometric interface includes a digital camera.

23. A system for verifying a credential for conveying verified information about an individual, wherein the system includes:
at least one acquisition interface operative to acquire:
a biometric associated with the credential, and
a verification code associated with the credential, and
an app responsive the interface and operative to verify that the acquired biometric matches the acquired verification code.

24. The system of claim 23 wherein the system is implemented with a smartphone and the acquisition interface includes a digital camera.
